(19) United States
(12) Patent Application Publication
(10) Pub. No.: US 2005/0282911 A1
(43) Pub. Date: Dec. 22, 2005

(54) Memantine for the prevention or reduction of suicidality and for treatment of major depression associated with suicidality

(76) Inventors: Heikki Hakkarainen, Madison, NJ (US); Scott McDonald, Chatham, NJ (US); Charles E. Flicker, Merritt Island, FL (US)

Correspondence Address: DARBY & DARBY P.C., P. O. BOX 5257, NEW YORK, NY, 10150-5257, US

(21) Appl. No.: 11/006,487

(22) Filed: Dec. 6, 2004

Related U.S. Application Data
(60) Provisional application No. 60/527,268, filed on Dec. 5, 2003.

(51) Int. Cl. A61K 31/13

(52) U.S. Cl. 514/662

(57) ABSTRACT

The present invention relates to the treatment of major depressive disorder (MDD), and the prevention of suicidality associated therewith, using the uncompetitive NMDA receptor antagonist memantine.

This application claims priority from U.S. provisional application Ser. No. 60/527,268, filed on Dec. 5, 2003, herein incorporated in its entirety.

### FIELD OF THE INVENTION

The present invention relates to the treatment of major depressive disorder (MDD), and the reduction or prevention of suicidality associated therewith, using the uncompetitive NMDA receptor antagonist memantine.

### BACKGROUND OF THE INVENTION

Major depressive disorder (MDD) is associated with high mortality. According to the Diagnostic and Statistical Manual of Mental Disorders IV (DSM IV-Branden/Hill, published by the American Psychiatric Association, Washington D.C., 1994), 15% of individuals with sever MDD die by suicide. This rate increases by almost fourfold in individuals who are over age 55. Risk of suicide in MDD is especially high in individuals with psychotic features of MDD, a history of previous suicide attempts, a family history of suicides, or concurrent substance abuse. Attempted and completed suicide is correlated with reduced serotonin metabolites in the cerebrospinal fluid at autopsy, but the degree to which this is correlated with the severity of other depressive symptoms is unclear (Asberg et al., Arch Gen Psychiatry. 1976; 33:1193-1197). Greater levels of suicidal ideation have been correlated with increased risk for a relapse marked by persistent mild depressive symptoms in relapse, rather than fully asymptomatic status (Judd et al., Am J Psychiatry. 2000; 157:1501-1504).

Major depressive disorder can begin at any age, with average onset in the mid-20's. According to DSM-IV, women have a 10-25% chance of having at least one MDD episode, while men have a 5-12% chance. MDD is characterized by a period of at least two weeks of a depressed mood or loss of interest or pleasure in activities, and includes additional symptoms such as changes in appetite or weight, sleep, and psychomotor activities; decreased energy; feelings of worthlessness or guilt; difficulty thinking, concentrating or making decisions, or recurrent thoughts of suicide. Individuals with MDD often present with tearfulness, irritability, brooding, anxiety, excessive worry, phobias, and complaints of physical pain. In addition, there is evidence for a familial pattern of MDD being 1-3 times more common among first-degree biological relatives.

Other disorders that frequently occur concomitantly with MDD include panic disorder, obsessive-compulsive disorder, anorexia nervosa, bulimia nervosa, and borderline personality disorder.

### Current Treatments

The primary approach to the treatment of major depressive disorder in the United States is the use of the selective serotonin reuptake inhibitors (SSRIs). Recently, the use of selective norepinephrine reuptake inhibitors (NARIs), and dual SSRI/NARIs, called SNRIs, has also become prevalent. 3-chloroimipramine, which inhibits both serotonin and norepinephrine reuptake, has been extensively used as an antidepressant in Europe and Canada. Other compounds which are of current interest or have been examined as antidepressants include fluvoxamine, citalopram, escitalopram, zimeldine, sertraline, bupropion and nomifensine. Fluvoxamine facilitates serotoninergic neurotransmission via potent and selective inhibition of serotonin reuptake into presynaptic neurons. Reboxetine is a selective norepinephrine reuptake inhibitor with potential utility in the treatment of severe depression. Other compounds, such as milnacipran, block both 5-HT and norepinephrine reuptake.

Many of these compounds have adverse side effects when administered at therapeutic levels. Potential side effects of SNRIs include nausea, headache, dry mouth, sedation, and tremors. The adverse effects occurring most frequently during treatment with selective SSRIs such as fluvoxamine are gastrointestinal disturbances, such as nausea, diarrhea/loose stools, constipation, with an incidence of 6 to 37% (Leonard, Drugs 1992, 43 (Suppl. 2): 3-9), and sexual dysfunction. Nausea is the main adverse effect in terms of incidence. These adverse effects, although mild to moderate in severity, deter some patients from treatment with SSRIs and SNRIs.

Suicide. Treatment of subjects at risk of committing suicide with antidepressants is considered to be beneficial because such subjects typically suffer from depression. However, certain clinicians and investigators believe that SSRI administration to such subjects has been linked to increased suicidality, based on meta-analyses of efficacy and epidemiological studies (Healy, J. Psychiatry Neurosci 2003; 28(5): 337-7). It has been hypothesized that the during the initial 2-3 week latency period prior to onset of anti-depressant activity of the SSRIs, there are increased neurotransmitter concentrations at neuronal synapses, including serotonin. This neurotransmitter increase can result in increased anxiety and agitation (i.e., akathisia) or more generally, irritability during the latency period, which may increase the risk of a suicide attempt. It has been reported that about 5% of patients (not suicidal patients but patients under treatment with SSRIs for depression) drop out of SSRI trials due to akathisia during this period (Healy, supra).

Accordingly, there is a need in the art to identify and develop therapeutic antidepressants having a different mechanism of activity than the SSRIs, and a faster onset of activity (and hence, a decreased latency period) than the SSRIs, in order to prevent or reduce suicidality in individuals suffering from suicidality.

### NMDA Receptor Antagonists

The N-methyl-D-aspartate (NMDA) receptor is a postsynaptic, ionotropic receptor which is responsive to the amino acids glutamate and glycine, and the synthetic compound NMDA. The NMDA receptor controls the flow of both divalent (Ca++) and monovalent (Na+, K+) ions into the postsynaptic neural cell through a receptor associated channel (Foster et al., Nature 1987; 329:395-396; Mayer et al., Trends in Pharmacol. Sci. 1990; 11:254-260). There is also a strychnine insensitive glycine binding site proximate to the NMDA/glutamate/aspartate binding site. It has been shown that glycine site agonists are necessary for channel function and that low intrinsic activity partial agonists, such as HA-966 (3-amino-1-hydroxypyrrolid-2-one; Merck) behave as functional NMDA antagonists in the presence of sufficient agonist.

U.S. Pat. No. 5,086,072, issued to Trullas et al., described the use of 1-aminocyclopropanecarboxylic acid (ACPC), which was thought to be a functional antagonist of the NMDA site via its activity as a partial agonist of the strychnine-insensitive glycine binding site, to treat mood disorders including major depression, bipolar disorder, dysthymia and seasonal affective disorder. It was also therein described that intraperitoneally-administered ACPC, via this functional antagonism, mimicked the actions of clinically effective antidepressants in animal models. However, it was subsequently demonstrated that sustained exposure to ACPC in fact attenuated its protective effects and increased the receptors' sensitivity to glutamate (Fossom et al., Mol. Pharmacol. 1995; 48: 981-87), and that ACPC is actually a full glycine agonist (Nathum-Levy et al., Mol. Pharmacol. 1999; 56: 1207-18).

International (PCT) Application WO 00/02551, to Mueller et al., described novel compounds active at both the serotonin reuptake site and the NMDA receptor (i.e., inhibition of both sites) that can be used to treat different types of disorders such as depression, obsessive-compulsive disorders (OCD), sleep disorders, sexual dysfunction, and eating disorders. According to this PCT, potent activity at the serotonin reuptake site was favored, while an intermediate activity at the NMDA receptor was favored. Too potent an activity at the NMDA receptor is less preferred because of possible PCP-like side effects.

A number of preclinical experiments have been reported as evidence that glutamate and the NMDA receptor may be involved in the etiology of depressive disorders (Skolnick, Eur J. Pharmacol. 1999; 375: 31-40; and Skolnick et al., Pharmacopsychiatry. 1996; 29:1, 23-6). NMDA receptor antagonists have been shown to exhibit antidepressant like activity in animal models of depression (Rogoz et al., Neuropharmacology. 2002; 42(8): 1024-30). Memantine, a moderate affinity, uncompetitive N-methyl-D-aspartate (NMDA) receptor antagonist, reduces glutamatergic output via open-channel block of the NMDA receptor-associated ion channel thereby reducing or preventing neuronal damage from excitotoxicity. See, e.g., U.S. Pat. Nos. 6,071,966; 6,034,134; and 5,061,703, all incorporated herein by reference. Memantine is also widely used for the treatment of Parkinson's disease, dementia, and spasticity in Germany, and has been approved for the treatment of moderately severe to severe Alzheimer's disease in the European Union and in moderate to severe Alzheimer's disease the United States. It is also currently being evaluated in the United States in clinical studies of patients with painful diabetic neuropathy.

### SUMMARY OF THE INVENTION

The present invention provides a method of treating major depressive disorder (MDD) using memantine.

The present invention also provides a method of preventing or reducing suicide risk by administering memantine to a subject suffering from suicidality.

### BRIEF DESCRIPTION OF THE DRAWINGS

FIG. 1 compares treatment with memantine, citalopram and escitalopram using the Montgomery Asberg Depression Rating Scale (MADRS) to demonstrate change from baseline in MDD patients.

FIG. 2 compares treatment with memantine, citalopram and escitalopram using the Hamilton Depression Rating Scale (HDRS) to demonstrate change from baseline in MDD patients.

FIG. 3 is a graph showing the CGI-S change from baseline as a function of treatment time.

FIG. 4 is a bar graph showing CGI-I Response as a function of treatment time.

### DETAILED DESCRIPTION

The present invention is based on results from an open-label, flexible-dose, 12-week study of memantine in eight patients with MDD. This study was designed to evaluate the safety and efficacy of memantine in the treatment of major depressive disorder. Unexpectedly, the results demonstrated a rapid-onset therapeutic benefit in the treatment of MDD (after 1 week). Not only is this an indication that memantine would be particularly useful in treating persons with major depressive disorder wherein a rapid onset of relief is indicated, but it also supports a utility for memantine to treat suicidality, as will be explained below.

### Definitions

“Memantine” refers to 1-amino-3,5-dimethyladamantane hydrochloride. In the United States, the trade name for memantine is Namenda®, in Germany as Akatinol and Auxura, and Ebixa in the European Union.

“Major depressive disorder”, or “MDD”, is described above and also according to the criteria in DSM-IV, incorporated herein by reference. The DSM-IV criteria can be used to diagnose patients as suffering from depression. The term also contemplates all diseases and conditions which are associated with MDD, including those classified in the IDC-10 (World Health Organization) and DSM-IV rating scales.

The term “treating”, as used herein, refers to reversing, alleviating, inhibiting the progress of, or preventing at least one overt symptomatic manifestation of the disorder or condition to which such term applies, or one or more symptoms of such disorder or condition. The term “treatment”, as used herein, refers to the act of treating, as “treating” is defined immediately above. For the present invention, the term “treat” means to alleviate or eliminate one or more of the symptoms, behavior or events associated with MDD, or with suicidality (e.g., reduction in suicidal ideation).

The term “prevention” refers to the prevention of the onset of a disease, which means to prophylactically interfere with a pathological mechanism that results in a disease or undesirable effect. In the context of the present invention, such a pathological mechanism can be prevention of symptoms associated with MDD, such as but not limited to those as identified using the DSM-IV diagnostic criteria, the HAM-D criteria, the MADRS, or the IDC-10 criteria. The term “prevent” also means prophylactic use of memantine in a subject to avert behavior or events associated with MDD or with suicidality. Subjects having or at risk for developing MDD, such as those with a familial patterns of MDD, can be identified by a diagnostic or prognostic assays according to the ordinary skill in the art.

The term “suicide” refers to completed suicide.

The term “suicidality” refers to a condition or disorder characterized by the occurrence, particularly the repeated occurrence, of suicidal thoughts (“suicidal ideation”) or suicidal impulse (loss of impulse control) or behavior. Suicidal behavior may include acts of self-harm with a fatal (“completed suicide”) or non-fatal (“attempted suicide”) outcome.

The term “suicidal ideation” more specifically refers to having thoughts of suicide or of taking action to end one's own life. Suicidal ideation includes all thoughts of suicide, both when the thoughts include a plan to commit suicide and when they do not include a plan.

The term “therapeutically effective amount” is used herein to mean an amount or dose of memantine that is effective to ameliorate or prevent a symptom, behavior or event associated with MDD or suicidality or suicidal ideation. Alternatively, a therapeutically effective amount is sufficient to cause an improvement in a clinically significant condition or parameter associated with MDD in an individual in need thereof. Such symptoms, behaviors or events are described above and in DSM-IV.

The terms, “about” and “approximately” shall generally mean an acceptable degree of error or variation for the quantity measured given the nature or precision of the measurements. Typical, exemplary degrees of error or variation are within 20 percent (%), preferably within 10%, and more preferably within 5% of a given value or range of values. Numerical quantities given herein are approximate unless stated otherwise, meaning that the term “about” or “approximately” can be inferred when not expressly stated.

### Dosage and Administration

Memantine (NAMENDA™) is commercially available as the hydrochloride salt in 5 or 10 mg film-coated tablets. However, according to the present invention, the dosage form of memantine may be a solid, semisolid or liquid formulation. Formulation of memantine in semi-solid or liquid form is within the skill of the art, as the active ingredient is highly soluble in aqueous media. Usually the active substance, i.e., memantine, will constitute between 0.1 and 99% by weight of the formulation, more specifically between 0.5 and 20% by weight for formulations intended for injection and between 0.2 and 50% by weight for formulations suitable for oral administration.

The pharmaceutical formulation comprises the active ingredients, optionally in association with adjuvants, diluents, excipients and/or inert carriers.

To produce pharmaceutical formulations of memantine in the form of dosage units for oral application, the memantine (and any additional compounds) may be mixed with a solid excipient, e.g., lactose, saccharose, sorbitol, mannitol, starches such as potato starch, corn starch or amylopectin, cellulose derivatives, a binder such as gelatine or polyvinylpyrrolidone, disintegrants e.g., sodium starch glycolate, crosslinked PVP, cross-carmellose sodium and a lubricant such as magnesium stearate, calcium stearate, polyethylene glycol, waxes, paraffin, and the like, and then compressed into tablets. If coated tablets are required, the cores, prepared as described above, may be coated with a concentrated sugar solution which may contain e.g., gum arabic, gelatine, talcum, titanium dioxide, and the like. Alternatively, the tablets can be coated with a polymer known to one skilled in the art, wherein the polymer is dissolved in a readily volatile organic solvent or mixture of organic solvents. Dyestuffs may be added to these coatings in order to readily distinguish between tablets containing different active substances or different amounts of the active compounds.

For the formulation of soft gelatin capsules, the active substances may be admixed with e.g., a vegetable oil or poly-ethylene glycol. Hard gelatin capsules may contain granules of the active substances using either the above mentioned excipients for tablets e.g., lactose, saccharose, sorbitol, mannitol, starches (e.g., potato starch, corn starch or amylopectin), cellulose derivatives or gelatine. Also liquids or semisolids of the drug can be filled into hard gelatine capsules.

Dosage units for rectal application can be solutions or suspensions or can be prepared in the form of suppositories comprising the active substances in a mixture with a neutral fatty base, or gelatin rectal capsules comprising the active substances in admixture with vegetable oil or paraffin oil. Liquid formulations for oral application may be in the form of syrups or suspensions, for example solutions containing from about 0.2% to about 20% by weight of the active substances herein described, the balance being sugar and mixture of ethanol, water, glycerol and propylene glycol. Optionally such liquid formulations may contain coloring agents, flavoring agents, saccharine and carboxymethyl-cellulose as a thickening agent or other excipients known to a person skilled in the art.

Solutions for parenteral applications by injection can be prepared in an aqueous solution of a water-soluble pharmaceutically acceptable salt of the active substances, preferably in a concentration of from about 0.5% to about 10% by weight. These solutions may also contain stabilizing agents and/or buffering agents and may conveniently be provided in various dosage unit ampoules.

Suitable daily doses of the active compounds, i.e., memantine, in therapeutic treatment of humans are about 0.01-10 mg/kg bodyweight on peroral administration and 0.001-10 mg/kg bodyweight on parenteral administration.

In a preferred embodiment, memantine will be administered within the range from about 5 mg to about 100 mg per day, preferably, from about 20 to about 40 mg per day.

Treatment duration can be short-term, e.g., several weeks (for example 10-14 weeks), or long-term until the attending physician deems further administration no longer is necessary.

### EXAMPLE

The invention is also described by means of a particular example or examples. However, the use of such examples is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

### Example 1

### Evaluation of Onset of Efficacy of Memantine on Symptoms and Behavior Associated with Major Depressive Disorder

The present study was a single-center, open-label, flexible dose, 12-week study designed to provide a preliminary assessment of the efficacy and safety of memantine in patients with major depressive disorder (MDD). In order to assess the efficacy of treatment on depressive symptomatology, the primary efficacy assessment was the Montgomery Depression Rating Scale (MADRS). Secondary efficacy assessments included the Hamilton Depression Rating Scale (HAM-D), the Clinical Global Impressions—Severity Scale (CGI-S), the Clinical Global Impressions—Improvement Scale (CGI-I), the Patient Global Evaluation (PGE), and the Quality of Life Scale (QOL).

### Methods

Study Design. The study was designed as a single-center, open-label, flexible dose 12-week study. Memantine was to be administered at 20 mg/day (10 mg b.i.d.) (titrated over a 4 week period), and, if warranted, up-titrated to a maximum of 40 mg/day (20 mg b.i.d.) (titrated in increments of 10 mg/day after Week 4).

Entrance Criteria. Criteria for enrollment were as follows: (i) male or female outpatients between 18 and 80 years of age at screening; (ii) diagnosis of MDD consistent with DSM-IV; (iii) Montgomery Asberg Depression Rating Scale (MADRS) score of 22 or greater; and CGI severity score of 4 or greater. None of the patients had attempted suicide or were diagnosed as being at risk for committing suicide.

Endpoints. The primary endpoint was improvement according to Montgomery Asberg Depression Rating Scale (MADRS). Secondary endpoints were improvements according to the Hamilton Depression Rating Scale (HAM-D), the Clinical Global Impressions-Severity Scale (CGI-S), the Clinical Global Impressions-Improvement Scale (CGI-I), Patient Global Evaluation (PGE), and Quality of Life Scale (QOL).

MADRS. MADRS is either self-administered (MADRS-S) or interviewer administered evaluation of symptoms of depression in adults. MADRS evaluates ten areas of depressive symptomatology: apparent sadness, reported sadness, inner tension, reduced sleep, reduced appetite, concentration difficulties, lassitude, inability to feel, pessimistic thoughts, and suicidal thoughts. Each area is rated on a seven-point scale (0-6).

MADRS was administered to each of the study participants at baseline, and weeks 1, 2, 3, 4, 6, 8, 10 and 12.

HAM-D. HAM-D criteria were assessed at weeks 1, 2, 4, 8 and 12. HAM-D is a 24-item scale that evaluates depressed mood, vegetative and cognitive symptoms of depression, and comorbid anxiety symptoms. It provides ratings on current DSM-IV symptoms of depression, with the exceptions of hypersomnia, increased appetite, and concentration/indecision. There are several ways of analyzing HAM-D. According to one analysis, the first 17-items are rated on either a 5-point (0-4) or a 3-point (0-2) scale. In general, the 5-point scale items use a rating of 0=absent; 1=doubtful to mild; 2=mild to moderate; 3=moderate to severe; 4=very severe. A rating of 4 is usually reserved for extreme symptoms. The 3-point scale items used a rating of 0=absent; 1=probable or mild; 2=definite. The second analysis uses the same scale to rate the first 21 items, and the third analysis uses the scale to rate all 24 items. All three analyses were used in the present study and used statistically in the results.

Response to medication (i.e., memantine) was defined as a reduction in the HAM-D 24 score of 50%, while remission was defined as a reduction in the HAM-D total score to 7 or less.

DSM-IV. DSM-IV diagnostic criteria were determined at week 1 and again at the end of the study. The DSM-IV checklist consists of 9 criteria for MDD as follows: a) depressed mood most of the day, subjectively or observed by others; b) markedly diminished interest or pleasure in all or almost all activities most of the day (subjective or objective); c) significant weight loss or weight gain (more than 5% in a month), or a decrease or increase in appetite nearly every day; d) insomnia or hypersomnia nearly every day; e) psychomoter agitation or retardation nearly every day (as observed by others); f) fatigue or loss of energy nearly every day; g) feelings of worthlessness or exessive or inappropriate guilt nearly every day; h) diminished ability to think or concentrate, or indecisiveness, nearly every day (subjective or objective); and i) recurrent thoughts of death, recurrent suicidal ideation without a specific plan, or a suicide attempt or a specific plan for committing suicide.

CGI-I and CGI-S. Clinical Global impression of Change (CGI-I) scores were assessed by the participants at baseline and visits 8 and 12. This assesses the patient's impression of his/her change. Clinical Global impression of Severity (CGI-S) were rated by the clinician at baseline, and at visits 8 and 12. This assesses the physician's impression of totality of response, including information about functioning and impairment, as well as relief of symptoms, from baseline.

Subset analyses. In addition, several items on the HAM-D and/or MADRS and/or DSM-IV checklist were considered in combinations of several items, as measures of change.

The combination HAM-D items were combinations of items 2 (guilt), 3 (suicide), 9 (agitation), 19 (depersonalizaton and derealization) and 21 (obsessive and compulsive symptoms). This combination is referred to as the ECDEU cognitive disturbance factor, and is a measure of cognitive disturbance.

To further evaluate effects of memantine on cognition, a combination of HAM-D item 8 (retardation) was combined with MADRS item 6 (concentration).

Other HAM-D combinations assessed were items 1 (depressed mood), 2 (guilt), 7 (work and activities), 8 (retardation and concentration), 10 (psychic anxiety) and 13 (agitation), referred to as the Bech Melancholia criteria; items 10, 11 (somatic anxiety), 12 (gastrointestinal somatic symptoms), 13, 15 (hypochondriasis), and 17 (insight regarding illness), known as the ECDEU anxiety factor criteria; items 1, 7, 8, and 14, as a measure of psychomotor retardation; and items 4-6 (insomnia-early, middle and late) as a measure of insomnia.

Statistics. The efficacy analyses were based on the ITT population using both last observation carried forward (LOCF) and observed cases (OC) approaches. For each of the parameters, descriptive statistics were presented for the actual values and change from baseline by visit. For categorical variables, frequency distributions were presented.

Patient Demographics and Baseline Characteristics. Seven of the eight patients were female. The mean age was 42 years (range: 22-71 years old). All patients were Caucasian. All patients had prior treatment with antidepressants. Seven of the eight patients had recurrent depression. The duration of major depressive disorder ranged from 2 to 43 years. At baseline, the mean MADRS score was 32 and the mean HAM-D score was 30. These scores are indicative of a population with severe depression.

Drug Treatment. All eight patients were initially given 5 mg/day and titrated over a 3-week period to a minimum dose of 20 mg/day. Patients with an unsatisfactory therapeutic response (CGI-I score greater than 2) could increase to a maximum of 40 mg/day (2 patients, 30 mg/day and 40 mg/day, respectively). One patient was titrated to 30 mg/day after Week 8, and two patients were titrated to 40 mg/day after Week 10. The mean treatment duration was 82 days (range: 57-86 days) and the mean daily dose was 18.1 mg/day.

All eight patients enrolled in the study received study medication and had at least one post baseline efficacy assessment of the primary efficacy variable, MADRS. Seven of the eight patients completed the study. One patient (#19005) was lost to follow-up after Week 8 (57 days).

Safety. Safety was assessed throughout the course of the study by monitoring vital signs and spontaneously reported adverse events.

### Results

Significant improvement from baseline to Week 12 for memantine treatment was observed on the primary and all secondary efficacy variables using both the LOCF and OC approaches (see Table 1, below).

TABLE 1
Mean Change from Baseline in Efficacy Assessments

| | MADRS | | HAM-D | | CGI-S | | CGI-I* | |
|---|---|---|---|---|---|---|---|---|
| | LOCF | OC | LOCF | OC | LOCF | OC | LOCF | OC |
| Baseline | 31.9 | 31.9 | 30.0 | 30.0 | 4.3 | 4.3 | — | — |
| Week 1 | −7.9 | −10.5 | −6.1 | −8.2 | −0.5 | −0.7 | 2.7 | 2.7 |
| Week 2 | −12.1 | −12.1 | −11.8 | −11.8 | −0.6 | −0.6 | 2.8 | 2.8 |
| Week 3 | −14.3 | −14.7 | — | — | −0.9 | −1.0 | 2.4 | 2.3 |
| Week 4 | −15.6 | −17.0 | −16.8 | −20.8 | −1.3 | −1.4 | 2.3 | 2.1 |
| Week 6 | −15.0 | −15.0 | — | — | −1.3 | −1.3 | 2.4 | 2.4 |
| Week 8 | −18.6 | −20.4 | −16.8 | −19.0 | −1.5 | −1.6 | 2.0 | 1.9 |
| Week 10 | −18.5 | −16.7 | — | — | −1.8 | −1.6 | 2.1 | 2.3 |
| Week 12 | −18.5 | −16.7 | −17.8 | −16.0 | −1.5 | −1.3 | 2.1 | 2.3 |

ITT Population.
*CGI-I mean score at each week.

At week 12, the mean change from baseline to endpoint was about 18.5 on the MADRS and about 17.8 on the HAM-D, with 62.5% of patients meeting criteria as CGI-I responders.

In addition, the reduction in MADRS and HAM-D (17-, 21-, and 24-question versions) at endpoint by LOCF analyses, of a magnitude of approximately 18 points, was greater than would be expected for 8 weeks of drug exposure to a proven SSRI, escitalopram (Burke et al., J Clin Psychiatry. 2002; 63 (4): 331-6). Further, much of the therapeutic effect appeared to occur even before the full maximal dose was achieved for each patient (i.e., by Week 4), suggesting an unusually rapid onset of effect.

Specific items of HAM-D chosen for analysis (items 1, 2, 3 and 7) also showed a positive change with memantine administration.

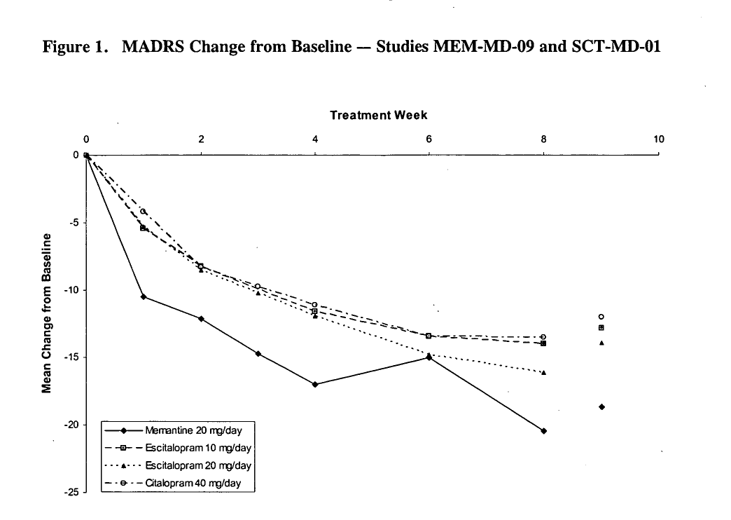

FIG. 1 presents the change from baseline in the MADRS by visit (through Week 8), by treatment group, for studies MEM-MD-09 (present study) and SCT-MD-01, a prior study. Study SCT-MD-01 was an 8-week fixed dose study that compared 10 mg/day citalopram and 20 mg/day escitalopram, to placebo and to 40 mg/day citalopram in outpatients. Escitalopram and citalopram at the doses tested are established treatments for use in patients with MDD.

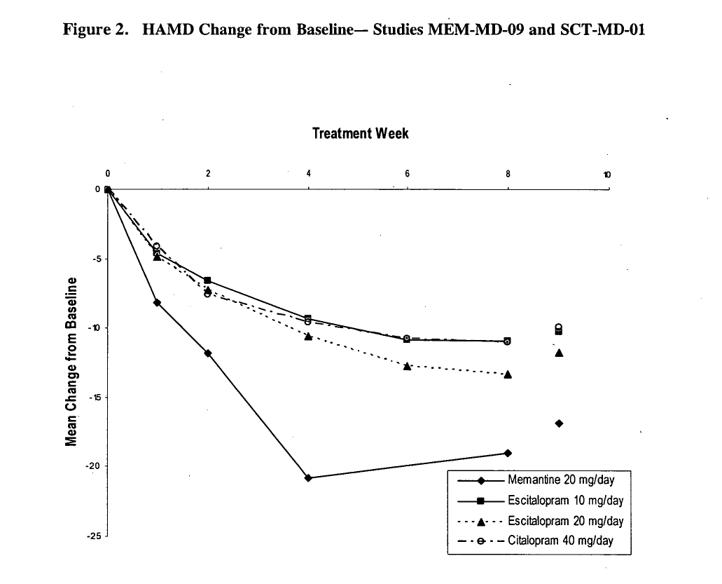

FIG. 2 presents the change from baseline in the HAM-D by visit (through Week 8), by treatment group for studies MEM-MD-09 and SCT-MD-01.

By the end of Week 8, 5 of the 7 patients responded to treatment, as measured by the MADRS and the HAM-D (responder defined as 50% improvement from baseline), and 6 of 7 patients were considered as “Very Much Improved” or “Much Improved” as measured by the CGI-I. Particularly striking was the rapid onset of relief which was marked already at the first assessment (one week). This is much faster than that measured for citalopram and escitalopram, the latter being considered the fastest onset of any SSRI.

This short latency period makes memantine a particularly suitable treatment for suicidality, as rapid relief from suicidal ideation or behavior is particularly desirable in such a patient population. Moreover, this feature makes memantine a particularly suitable treatment for patients who are afflicted with both suicidality and major depression, for whom physicians may have been reluctant to prescribe antidepressants because of the relatively long latency period, and because of reports that some SSRIs may contribute to suicidal ideation or behavior. The rapid onset of memantine coupled with its non-SSRI mode of action fills a perceived need in the art.

DSM-IV checklist. There was a reduction in the degree of symptomology in all DSM-IV categories, with a complete remission of symptoms in all patients in the categories of appetite and agitation/retardation.

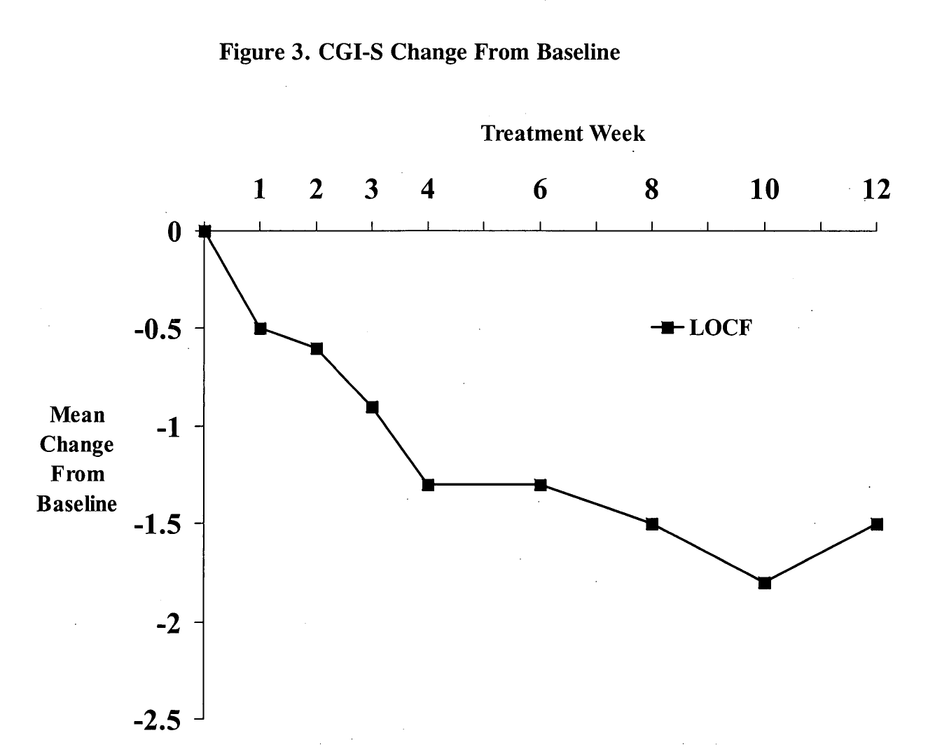

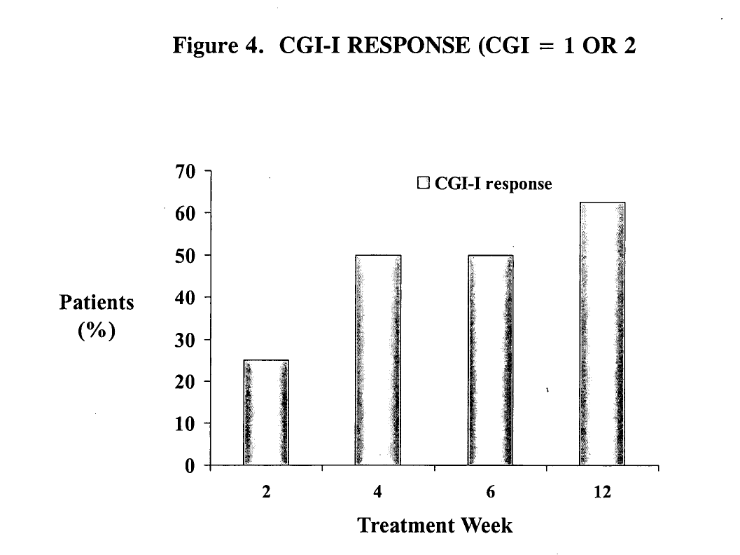

CGI-I and CGI-S. After 12 weeks, there was an overall impression of improvement as measured using CGI-I in all patients with the exception of one (FIG. 3). Similarly, there was a marked shift during the 12 week period of the trial; patients who were ranked as moderately to severely ill improved to “mildly depressed” or to “not depressed” (with the exception of one patient—FIG. 4).

Subsets

Cognitive disturbance. Although no direct measures of cognitive functioning were included, symptoms that might be the result of such a change were assessed. Each independent measure for HAM-D item 8 (retardation and concentration), MADRS item 6 (concentration difficulties), and DSM-IV criterion “h” (diminished ability to think or concentrate) improved during the 12 week treatment period.

Similarly, each measure in the HAM-D ECDEU combination, as a measure of cognition, improved with memantine treatment over the 12 week period. However, it is possible that the changes are secondary to the improvement in depressive mood and not to improvement in cognition per se.

Melancholy, anxiety, psychomotor retardation and insomnia. An improvement at week 12 from baseline was shown for all of the HAM-D item combinations specific for these criteria.

Suicide. A steady improvement in the score of item 3 of HAM-D was demonstrated throughout visit 8. One patient dropped out of the study during this time, asserting maximal improvement on this item. This further supports the suitability of memantine for treating suicidality.

Appetite. The overall scores on the two appetite items, HAM-D 12 and MADRS item 5 both showed a decrease from baseline, as did the appetite item “c” from the DSM-IV criterion list.

Safety and Adverse Events. Memantine 20-40 mg/day was safe and well tolerated. No deaths, serious adverse events or discontinuations due to adverse events were reported. The incidence of treatment emergent adverse events (TEAE) patients are provided in Table 2, below. There were no safety findings of concern for clinical laboratory parameters, vital signs or ECG values.

TABLE 2
Number of Patients Reporting Treatment Emergent Adverse Events
by Preferred Term

| Adverse Event Preferred Term | Memantine (N = 8) |
|---|---|
| Patients with at least one TEAE | 8 |
| Influenza-like Symptoms | 2 |
| Dizziness | 2 |
| Headache | 2 |
| Somnolence | 3 |
| Anxiety | 2 |
| Amnesia | 2 |
| Insomnia | 2 |

Safety Population (events reported by 2 or more patients).

### Conclusions

This study demonstrates significant improvement in MDD symptomology by each of four different scales with memantine treatment, which was observed during the first assessment one week following treatment, and continued for the duration of the study. The results were robust as improvement was consistently observed across all the primary and secondary efficacy parameters including the MADRS, the HAM-D, and the CGI. In addition, the rapid onset of effect occurred prior to the completion of the 4-week titration period and the antidepressant effect of memantine appeared considerably faster than that reported for other proven antidepressants, notably citalopram and escitalopram.

In summary, the conclusion drawn from this study was that memantine at doses of 20-40 mg/day, was safe and well tolerated, and demonstrated a larger magnitude and faster onset of overall therapeutic response compared to proven antidepressants citalopram and escitalopram. In particular, because of the faster onset of relief, and because of the non-SSRI mechanism of action, the study permits the inference that memantine is particularly suited for administration to subjects suffering from suicidality, and from major depression in patients also afflicted with suicidality or suicidal ideation. Additionally, the present data support the use of memantine, at least as initial therapy, in other cases of major depressive disorder which are in need of a rapidly effective treatment.

### Example 2

### Evaluation Memantine on Symptoms and Behavior Associated with Major Depressive Disorder in Alzheimer's Disease

The objective of this study (MEM-MD-17) was to evaluate the safety and efficacy of memantine and memantine in combination with escitalopram in patients with depression of Alzheimer's disease.

The clinical study was conducted in two phases —12-weeks of open-label treatment with memantine (MEM) followed by 12-weeks of randomized double-blind treatment with memantine+placebo (MEM+PBO) or memantine+escitalopram (MEM+SCT).

### Methods

Study design. Patients were started on memantine 5 mg/day which was increased to 10 mg/day at end of Week 1. The dose could be further increased to a maximum of 20 mg/day in patients with inadequate response in weekly increments of 5 mg. At end of Week 12, patients were randomized to one of the two treatment groups: MEM+PBO or MEM+SCT. The dose of memantine during the double-blind treatment period was fixed at the same level as at the end of Week 12 and the dose of escitalopram was fixed at 10 mg. Dose adjustments during the double-blind period were not permitted.

Patient Population. A total of 15 patients were enrolled in 2 sites. Three of the 15 patients discontinued during the open-label phase. The remaining 12 were randomized to double-blind treatment, 5 in the MEM+PBO treatment group, and 7 in the MEM+SCT treatment group. A total of 10 patients completed the study.

Endpoints. Several rating scales were used to assess efficacy for depression. The Cornell Scale for Depression in Dementia (CSDD); the HAMD; and the MADRS. The HAMD and MADRS have been described above.

CSDD. This is a 16 question scale which is subdivided into five sections: (A) Mood-Related Signs (questions 1-4); (B) Behavioral Disturbances (questions 5-8); (C) Physical Signs (questions 9-11); (D) Cyclic Functions (questions 12-15); and (E) Ideational Disturbances (questions 16-19). Question 16 is explicitly directed to suicidal ideation. The scoring system ranges from a=unable to evaluate; 0=absent; 1=mild to intermittent; and 2=severe.

Statistics. The efficacy analyses were based on the ITT population using both last observation carried forward (LOCF) and observed cases (OC) approaches. For each of the parameters, descriptive statistics were presented for the actual values and change from baseline by visit. For categorical variables, frequency distributions were presented.

### Results

The extent of improvement was similar to that observed in the previous open-label study with memantine in depressed patients (Study MEM-MD-09) which enrolled patients with moderate to severe depression.

Suicidality. Questions 16, 3 and 10, on the CSDD, HAMD, and MADRS respectively are specifically directed to suicide. Descriptive statistics are shown for suicide items in CSDD, HAMD and MADRS scales. Table 3 presents the mean change from baseline to Week 12 for all 15 patients who were enrolled in the study and received memantine. Table 4 presents the mean change from baseline at Week 12 and Week 24 for the 5 patients who received memantine throughout the study.

TABLE 3
Change from Baseline to Week 12 (LOCF) in Suicide Items ITT
Population Memantine (N = 15)

| | Baseline | Change at Week 12 |
|---|---|---|
| CSDD Item #16 | 0.4 | −0.3 |
| MADRS Item #10 | 0.9 | −0.6 |
| HAMD Item #3 | 0.5 | −0.5 |

TABLE 4
Change from Baseline to Week 24 in Suicide items
ITT Population Memantine (N = 5)

| | | Change at | Change at Week |
|---|---|---|---|
| | Baseline | Week 12 | 24 |
| CSDD item #16 | 0.4 | −0.4 | −0.3 |
| MADRS item #10 | 1.0 | −1.0 | −0.5 |
| HAMD item #3 | 0.8 | −0.8 | −0.5 |

Based on the change from Baseline at Weeks 12 and 24, memantine treatment led to modest but consistent numerical improvement in suicidality as measured by 3 different depression rating instruments.

As evidenced above, treatment with memantine for 12 and 24 weeks demonstrated a trend towards reduced suicidal thoughts and ideation.

Patents, patent applications, publications, product descriptions, and protocols are cited throughout this application, the disclosures of which are incorporated herein by reference in their entireties for all purposes.

1. A method of preventing or reducing suicidality comprising administering to an individual in need thereof an effective amount of memantine and a pharmaceutically acceptable carrier.

2. The method of claim 1, wherein memantine is administered in a range from about 5 to about 100 mg/day.

3. The method of claim 2, wherein memantine is administered in a range from about 20 to about 40 mg/day.

4. The method of claim 1 wherein the suicidality is suicidal ideation.

5. The method of claim 1, wherein the suicidality is suicidal behavior.

6. The method of claim 5, wherein the suicidal behavior comprises attempted suicide.

7. The method of claim 1 wherein the suicidality comprises suicidal impulse.

8. The method of claim 1 wherein the suicidality is accompanied by major depressive disorder.

9. The method of claim 1 wherein the reduction in suicidality is demonstrated after about 12 weeks of treatment.

10. The method of claim 1 wherein the reduction in suicidality is demonstrated after about 24 weeks of treatment.
